(19) United States
(12) Patent Application Publication
(10) Pub. No.: US 2007/0155663 A1
(43) Pub. Date: Jul. 5, 2007

(54) Use of chemokine receptor agonists for stem cell transplantation

(76) Inventors: Rudolf Richter, Hannover (DE); Reinhard Henschler, Rodermark (DE)

Correspondence Address: JACOBSON HOLMAN PLLC, 400 SEVENTH STREET N.W., SUITE 600, WASHINGTON, DC, 20004, US

(21) Appl. No.: 10/550,593

(22) PCT Filed: Mar. 24, 2004
(86) PCT No.: PCT/EP04/03115

§ 371 Date: Sep. 11, 2006

(30) Foreign Application Priority Data
Mar. 24, 2003 (EP) 03006552.8

(51) Int. Cl. A61K 38/17; A61N 5/00

(52) U.S. Cl. 514/12; 600/1

(57) ABSTRACT

A medicament comprising at least one agonist of receptors selected from the group consisting of the CCR3, CCR6 or CCR8 receptor or combinations thereof and a pharmaceutically acceptable carrier.

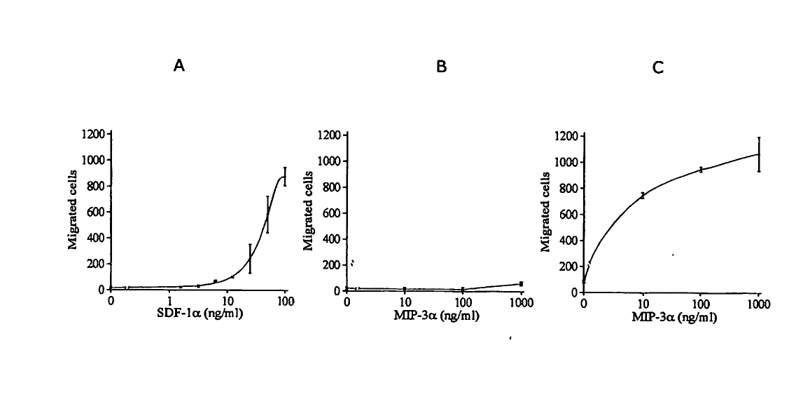

The invention pertains to a medicament comprising at least one agonist of receptors, the use of an agent for the manufacturing of a medicament for improving the homing of stem cells as well a method of improving the successful homing of hematopoietic stem cells.

### SUMMARY

Chemokines receptor agonists for chemokine receptors CCR3, CCR6 and CCR8 are found to increase the sensitivity of hematopoietic stem and progenitor cells to the SDF-1α signal. CCR3, CCR6 and CCR8 agonists were found to improve stem cell homing into the bone marrow during stem cell transplantation.

### FIELD OF THE INVENTION

The present invention relates to methods of using chemokines receptor agonists for chemokine receptors CCR3, CCR6 and CCR8 to improve stem cell homing into the bone marrow during stem cell transplantation.

### BACKGROUND OF THE INVENTION

Hematopoietic stem cells are rare primitive blood cell progenitors that have the capacity to self-replicate, to maintain a continuous source of regenerative cells, and to differentiate, to give rise to various morphologically recognizable precursors of blood cell lineages. These precursors are immature blood cells that cannot self-replicate and must differentiate into mature blood cells. Within the bone marrow microenvironment, the stem cells self-proliferate and actively maintain continuous production of all mature blood cell lineages throughout life.

Bone marrow transplantation is being increasingly used in humans as an effective therapy for an increasing number of diseases, including malignancies such as leukemias, lymphoma, myeloma and selected solid tumors as well as nonmalignant conditions such as aplastic anemias, immunological deficiencies and inborn errors of metabolism. The objective of BM transplantation is to provide the host with a healthy stem cell population that will differentiate into mature blood cells that replace deficient or pathologic cell lineages.

The source of the BM for transplantation may be autologous, syngeneic or allogeneic. Preferred are autologous BM or BM from HLA-matched siblings, but also BM from HLA-nonmatched donors is being used for transplantation.

Complicating factors in BM transplantation include graft rejection and graft-vs-host disease. Since donor T lymphocytes were found to cause GVHD in animals, one of the procedures to prevent or alleviate GVHD consists in. removing T cells from the donor BM before transplantation. This can be done by different techniques. Extensive use of T-cell depleted BM effectively prevented GVHD but, unfortunately, resulted in a high rate of graft rejection (10-15% in HLA-matched recipients and 50% in HLA-nonmatched recipients) or graft failure (as high as 50%).

Another problem in BM transplantation is the difficulty of achieving long-term successful engraftment also when no graft rejection or GVHD occurs. Nowadays, patients which were successfully transplanted have very low levels of stem cells and immature progenitors which generate mature blood cells, compared with healthy individuals.

Stem cells are functionally defined by their ability to home to the bone marrow and to durably repopulate transplanted recipients with both myeloid and lymphoid cells. The processes that mediate homing and engraftment of human stem cells to the bone marrow involve a complex interplay between cytokines, chemokines and adhesion molecules.

Much of our knowledge of the regulation and the hierarchical organization of the hematopoietic system derives from studies in the mouse wherein stem cells are identified and quantified in long-term reconstitution assays. In contrast, our knowledge of the biology of human hematopoiesis is limited, since it is mostly based on in characterize and quantify repopulating stem cells.

Intensive research is being carried out in order to understand the processes that mediate homing and engraftment of human stem cells to the bone marrow. Recently, several groups have established in vivo models for engraftment human stem cells, e.g. into immune deficient mice such as irradiated beige, nude, Xid (X-linked immune deficiency), SCID and non-obese diabetic SCID (NOD/SCID) mice, and in utero transplantation into sheep fetuses which resulted in successful multilineage engraftment of both myeloid and lymphoid cells.

Previously inventors have developed a functional in vivo assay primitive human SCID repopulating cells (SRCs) based on their ability to durably repopulate the bone marrow of intravenously transplanted SCID or NOD/SCID mice with high levels of both myeloid and lymphoid cells ([1, 2]). Kinetic experiments demonstrated that only a small fraction of the transplanted cells engrafted and that these cells repopulated the murine bone marrow by extensive proliferation and differentiation. Furthermore, the primitive human cells also retained the capacity to engraft secondary murine recipients [3]. Transplantation of populations enriched for CD34 and CD38 cell surface antigen expression, revealed that the phenotype of SRC is CD34+CD38− [2]. Other repopulating cells may exist since recent studies suggest that immature human CD34− cells and more differentiated CD34+CD38+ cells have some limited engraftment potential [4, 5].

Accumulating evidence indicates that stem cell homing to the bone marrow is a multistep process. The mechanisms involved in hematopoietic stem cell trafficking have been largely unknown for a long time.

During the past few years, the role of particular secreted (pg, cytokines) and cell-bound proteins (eg, adhesion molecules) in progenitor mobilization and homing has been recognized.[6-9] More recently, it has been shown that cytokines may play a central role in progenitor cell trafficking, particularly in stem cell homing to the bone marrow (BM).[9-12]. Interestingly, extravasation of mature leukocytes during inflammation and homing of immature progenitor and stem cells to the BM may at least partially depend on similar mechanisms [8]. Inflamed tissues and the hematopoietic microenvironment share similarities, such as expression of particular adhesion molecules (E-selectin, vascular cell adhesion molecule-1) on microvascular endothelium [13, 14].

Of particular interest for bone marrow engraftment are the chemokine stromal cell-derived factor-1 (SDF-1) and its receptor CXCR4. Treatment of human progenitor cells with antibodies to CXCR4 prevented engraftment into human severe combined immunodeficient (NOD/SCID) mice. In vitro CXCR4-dependent migration to SDF-1 of CD34+CD38−/low cells was found to correlate with in vivo engraftment and stem cell function [10]. Activation of CD34(+) cells with SDF-1a leads to firm adhesion and transendothelial migration, which is dependent on LFA-1/ICAM-1 (intracellular adhesion molecule-1) and VLA-4/VCAM-1 (vascular adhesion molecule-1). Furthermore, SDF-1-induced polarization and extravasation of CD34(+)/CXCR4(+) cells through the extracellular matrix underlining the endothelium is dependent on both VLA-4 and VLA-5[15].

In view of expanded approach to treatment of many severe diseases by hematopoietic stem cell transplantation, it is highly desirable to understand better the mechanism behind stem cell homing to the bone marrow and repopulation of transplanted hosts in order to obtain stem cells with higher rates of successful and long-term engraftment.

### SUMMARY OF THE INVENTION

According to the invention a medicament improves the homing of stem cells in a patient receiving a stem cell graft which medicament is comprising at least one agonist of receptors selected from the group consisting of the CCR3, CCR6 or CCR8 receptor or combinations thereof and a pharmaceutically acceptable carrier.

Subject matter of the invention is also the use of an agent for the manufacturing of a medicament for improving the homing of stem cells wherein the agent is at least one agonist of receptors selected from the group consisting of the CCR3, CCR6 or CCR8 receptor or combinations thereof.

In one embodiment of the use of the invention the agonist is used for treatment of progenitor and stem cells prior to transplantation.

In a further embodiment of the invention the agent is used for the transplantation of hematopoietic progenitor and stem cells, umbilical cord blood and placental stem and progenitor cells, liver stem and progenitor cells (oval cells), mesenchymal stem and progenitor cells, endothelial progenitor cells, skeletal muscle stem and progenitor cells (satellite cells), smooth muscle stem and progenitor cells, intestinal stem and progenitor cells, embryonic stem cells, and genetically modified embryonic stem cells, adult islet/beta stem- and progenitor cell, epidermal progenitor and stem cells, keratinocyte stem cells of cornea, skin and hair follicles, olfactory (bulb) stem and progenitor cells and side population cells from diverse adult tissues.

The use of the agent according to the invention increases the sensitivity of hematopoietic stem cells to SDF-1 induced cellular signals.

In particular the agent is used according to the invention for the treatment of leukemias, lymphoproliferative disorders, aplastic anemia, congenital disorders of the bone marrow, solid tumors, autoimmune disorders, inflammatory diseases, primary immunodeficiencies, primary systemic amyloidosis, systemic sclerosis, heart diseases, liver diseases, neurodegenerative diseases, multiple sclerosis, M. Parkinson, stroke, spinal cord injury diabetes mellitus, bone diseases, skin diseases, replacement therapy of the skin, retina or cornea, other congenital disorders, vessel diseases like atherosclerosis or cardiovascular disease.

In another embodiment of the invention a method of improving the successful homing of hematopoietic stem cells is disclosed by contacting the hematopoietic stem cells in vivo or ex vivo with an agent which is at least one agonist of receptors selected from the group consisting of the CCR3, CCR6 or CCR8 receptor or combinations thereof.

In a further embodiment of the invention a method of improving the successful homing of hematopoietic stem cells in a host patient is disclosed by applying into the patient which are receiving stem cell transplantation prior to and/or in the course of stem cell transplantation in vivo at least one agent which is an agonist of receptors selected from the group consisting of the CCR3, CCR6 or CCR8 receptor or combinations thereof.

In the method of the invention the host patient may not conditioned or the host patient is conditioned under sublethal, lethal, or supralethal conditions. In particular sublethal, lethal, or supralethal conditions include treatment with total body irradiation, optionally followed by treatment with myeloablative or immunosuppressive agents. The sublethal, lethal, or supralethal conditions include myeloablative or immunosuppressive treatment without total body irradiation. Typical examples of agonists for CCR3, CCR6, and CCR8 are shown in the Table

TABLE
Ligands, which regulate stem cell homing
in synergy with SDF-1α and CXCR4

| Receptor | Ligand |
|---|---|
| CCR3 | Eotaxin |
| | Eotaxin-2 |
| | Eotaxin-3 |
| | Hemofiltrate CC Chemokine-1 (HCC-1) |
| | Hemofiltrate CC Chemokine-2 (HCC-2) |
| | Macrophage Inflammatory Protein - 1α (MIP-1α) |
| | Regulated on Activation Normally T-Cell Express and |
| | Secreted (RANTES) |
| | Monocyte Chemoattractant Protein - 2 (MCP-2) |
| | Monocyte Chemoattractant Protein - 3 (MCP-3) |
| | Monocyte Chemoattractant Protein - 4 (MCP-4) |
| | 2-[(6-amino-2-benzothiazolyl)thio]-N-[1-[(3,4- |
| | dichlorylphenyl)-methyl]-4-piperidinyl]acetamide |
| CCR6 | Macrophage Inflammatory Protein - 3α (MIP-3α) |
| CCR8 | I309 |
| | Macrophage Inflammatory Protein - 1β (MIP-1β) |
| | LAG-1 |
| | Thymus and Activation Regulated Chemokine (TARC) |
| | viral Macrophage Inflammatory Protein - I (vMIP-I) |

Table: Ligands, which regulate stem cell homing in synergy with SDF-1α and CXCR4

The present investigation thus relates to a method for increasing the sensitivity of hematopoietic progenitor- and stem cells to migrate in response to CXCR4 activation and/or to increase the capability to adhere to stromal cells. In this aspect the present invention provides a method for increasing the sensitivity of hematopoietic stem and progenitor cells for use in clinical transplantation. The method is related to a pretreatment of transplantable hematopoietic progenitor- and stem cells with CCR3, CCR6, and CCR8 agonists prior to transplantation and/or to in vivo application of CCR3, CCR6, and CCR8 agonists to patients prior-, during, and/or subsequently to stem cell transplantation.

A further aspect of the invention relates to a method for transplantation of immature hematopoietic cells in patients. The patients need conditioning under sublethal, lethal or supralethal conditions, for example by total body irradiation (TBI) and/or by treatment with myeloablative and immunosupressive agents according to standard protocols. For example, a sublethal dose of irradiation is within the range of 3-7 Gy TBI, a lethal dose is within the range of 7-9.5 Gy TBI, and a supralethal dose is within the range of 9-16.5 Gy TBI. Examples of myeloablative agents are busulphan, dimethyl mileran and thiotepa, and of immunosupressive agents are prednisolone, methyl prednisolone, azathioprine, cyclophosphamide, cyclophosphamide, etc.

The method of the invention is suitable for the treatment of diseases curable by bone marrow transplantation such as malignant diseases, including leukemias, solid tumors, congenital or genetically-determined hematopoietic abnormalities, like severe combined immunodeficiency syndromes (SCID) including adenosine deaminase (ADA) deficiency, osteopetrosis, aplastic anemia, Gaucher's disease, thalassemia.

The present invention is further disclosed by the following non-limiting embodiments.

Modulation of homing mechanisms by preincubation with CCR3, -6, -8 agonists in vitro

For example enriched CD34+ progenitor cells from human cord blood, mobilized peripheral blood, or bone marrow are incubated with one of the CCR3, -6, -8 agonists typically in concentrations between 100 pM and 10 μM for a time period which is between 5 minutes and 12 hours.

The principle of the modulation of homing mechanisms by preincubation with CCR3, -6, -8 agonists is exemplified as follows.

[image]

After preincubation stem cells are transplanted into the patients preconditioned with chemotherapeutic regimen or with total body irradiation. Recovery of the hematopoietic system is monitored by the platelet and neutrophil blood counts.

Modulation of homing mechanisms by preincubation with CCR3, -6, -8 agonists in vivo can be performed as explained infra.

Prior to transplantation of hematopoietic stem cells patients receive conditioning by total body irradiation (TBI) and/or by treatment with myeloablative and immunosupressive agents according to standard protocols. 24 h to 0 h prior to stem cell transplantation patients start a continuous infusion of one of the CCR3, CCR6 or CCR8 agonists, reaching plasma concentrations between 100 pM and 10 μM of the agonist. 24 to 48 hours after preconditioning by chemotherapy or irradiation patients receive enriched CD34+ progenitor cells from human cord blood, mobilized peripheral blood, or bone marrow. These cells are either untreated or incubated with one of the CCR3, -6, -8 agonists in concentrations between 100 pM and 10 μM for a time period which is between 5 minutes and 12 hours. Recovery of the hematopoietic system is monitored by the platelet and neutrophil blood counts.

Figure: FDCP-Mix cells were subjected to in vitro chemotactic assays. Chemotaxis was assessed in 96-transwell chambers (Neuroprobe, Cabin John, MD) by using polyvinylpyrrolidone-free polycarbonate membranes (Nucleopore, Neuroprobe) with 5-μm pores. Four hundred microliters of IMDM medium was added to the bottom of the well, and was supplemented with varying concentrations of SDF-1α or MIP-3α (R&D Systems). 100 μl of IMDM medium containing 50.000 FDCP-Mix cells were added to the upper wells of the chemotaxis chamber. Additionally 100 μl of medium either with no supplement or supplemented with MIP-3α was added to the upper well. All assays were carried out in triplicate, and the migrated cells were counted in 4 randomly selected fields at 63-fold magnification after migration for 14 h.
(A) Chemotactic migration was induced by increasing concentrations of SDF-1a in the bottom well of the chemotaxis chamber.
(B) MIP-3a was subjected to the bottom well in concentrations of 10 to 1000 ng/ml medium. MIP-3a does not induce chemotactic migration of the FDCP-Mix progenitor cells.
(C) SDF-1a was subjected to the bottom well in a concentration of 10 ng/ml medium. Simultaneously FDCP-Mix progenitor cells were coincubated with MIP-3α in concentrations of 10 to 1000 ng/ml medium. In summary MIP-3α does increase the sensitivity the of the FDCP-Mix cells to migrate to SDF-1α. This effect was also identified for CCR3 receptor agonists Eotaxin, Eotaxin-2, Rantes, MCP-2, MCP-3, MCP-4, and CCR8 receptor agonist I-309.

### REFERENCES

1. Lapidot T, P. F., Doedens M, Murdoch B, Williams D E, Dick J E, Cytokine stimulation of multilineage hematopoiesis from immature human cells engrafted in SCID mice. Science, 1992. 255: p. 255.
2. Larochelle A, V. J., Hanenberg H, Wang J C, Bhatia M, Lapidot T, Moritz T, Murdoch B, Xiao X L, Kato I, Williams D A, Dick J E, Identification of primitive human hematopoietic cells capable of repopulating NOD/SCID mouse bone marrow: implications for gene therapy. Nat Med, 1996. 2: p. 1329-37.
3. Cashman J, B. K., Hogge D E, Eaves A C, Eaves C J, Sustained proliferation, multi-lineage differentiation and maintenance of primitive human haemopoietic cells in NOD/SCID mice transplanted with human cord blood. Br J Haematol, 1997. 98: p. 1026-36.
4. Zanjani E D, A.-P. G., Livingston A G, Flake A W, Ogawa M, Human bone marrow CD34− cells engraft in vivo and undergo multilineage expression that includes giving rise to CD34+ cells. Exp Hematol, 1998. 26: p. 353-60.
5. Conneally E, C. J., Petzer A, Eaves C, Expansion in vitro of transplantable human cord blood stem cells demonstrated using a quantitative assay of their lympho-myeloid repopulating activity in nonobese diabetic-scid/scid mice. Proc Nati Acad Sci U S A, 1997. 94: p. 9836-41.
6. Esmail D. Zanjani, A. W. F., Graça Almeida-Porada, Nam Tran, and Thalia Papayannopoulou, Homing of Human Cells in the Fetal Sheep Model: Modulation by Antibodies Activating or Inhibiting Very Late Activation Antigen-4-Dependent Function. Blood, 1999. 94: p. 2515-2522.
7. Greenberg A W, K. W., Hammer D A, Relationship between selectin-mediated rolling of hematopoietic stem and progenitor cells and progression in hematopoietic development. Blood, 2000. 95: p. 478-86.
8. Mohle R, B. F., Rafii S, Moore M A, Brugger W, Kanz L, Regulation of transendothelial migration of hematopoietic progenitor cells. Ann N Y Acad Sci, 1999. 872: p. 176-85.
9. Naiyer A J, J. D., Ahn J, Mohle R, Peichev M, Lam G, Silverstein R L, Moore M A, Rafli S, Stromal derived factor-1-induced chemokinesis of cord blood CD34(+) cells (long-term culture-initiating cells) through endothelial cells is mediated by E-selectin. Blood, 1999. 94: p. 4011-9.
10. Peled A, P. I., Kollet O, Magid M, Ponomaryov T, Byk T, Nagler A, Ben-Hur H, Many A, Shultz L, Lider O, Alon R, Zipori D, Lapidot T, Dependence of human stem cell engraftment and repopulation of NOD/SCID mice on CXCR4. Science, 1999. 283: p. 845-8.
11. Aiuti A, W. I., Bleul C, Springer T, Gutierrez-Ramos J C, The chemokine SDF-1 is a chemoattractant for human CD34+ hematopoietic progenitor cells and provides a new mechanism to explain the mobilization of CD34+ progenitors to peripheral blood. J Exp Med, 1997. 185: p. 111-20.
12. Mohle R, B. F., Rafii S, Moore M A, Brugger W, Kanz L, The chemokine receptor CXCR-4 is expressed on CD34+ hematopoietic progenitors and leukemic cells and mediates transendothelial migration induced by stromal cell-derived factor-1. Blood, 1998. 91: p. 4523-30.
13. Schweitzer K M, D. A., van der Valk P, Thijsen S F, Zevenbergen A, Theijsmeijer A P, van der Schoot C E, Langenhuijsen M M, Constitutive expression of E-selectin and vascular cell adhesion molecule-1 on endothelial cells of hematopoietic tissues. Am J Pathol, 1996. 148: p. 165-75.
14. Jacobsen K, K. J., Kincade P W, Osmond D G, Adhesion receptors on bone marrow stromal cells: in vivo expression of vascular cell adhesion molecule-1 by reticular cells and sinusoidal endothelium in normal and gamma-irradiated mice. Blood, 1996. 87: p. 73-82.
15. Peled A, K. O., Ponomaryov T, Petit I, Franitza S, Grabovsky V, Slav M M, Nagler A, Lider O, Alon R, Zipori D, Lapidot T, The chemokine SDF-1 activates the integrins LFA-1, VLA-4, and VLA-5 on immature human CD34(+) cells: role in transendothelial/stromal migration and engraftment of NOD/SCID mice. Blood, 2000. 95: p. 3289-96.

1. A medicament comprising at least one agonist of receptors selected from the group consisting of the CCR3, CCR6 or CCR8 receptor or combinations thereof and a pharmaceutically acceptable carrier.

2. The medicament according to claim 1 wherein the agonists is selected from the group consisting
of receptor CCR3: Eotaxin; Eotaxin-2; Eotaxin-3; Hemofiltrate CC-Chemokine-1 (HCC-1); Hemofiltrate CC Chemokine-2 (HCC-2); Macrophage Inflammatory Protein-1α (MIP-1α); Regulated on Activation Normally T-Cell Express and Secreted (RANTES); Monocyte Chemoattractant Protein-2 (MCP-2); Monocyte Chemoattractant Protein-3 (MCP-3); Monocyte Chemoattractant Protein-4 (MCP-4); 2-[(6-amino-2-benzothiazolyl)thio]-N-[1-[(3,4-dichlorylphenyl)methyl]-4-piperidinyl] acetamide;
of receptor CCR6: Macrophage Inflammatory Protein-3α (MIP-3α);
of receptor CCR8: I309; Macrophage Inflammatory Protein -1β (MIP-1β); LAG-1; Thymus and Activation Regulated Chemokine (TARC); viral Macrophage Inflammatory Protein-I (vMIP-I); as well as derivatives therof keeping their agonist abilities.

3. Use of an agent for the manufacturing of a medicament for improving the homing of stem cells wherein the agent is at least one agonist of receptors selected from the group consisting of the CCR3, CCR6 or CCR8 receptor or combinations thereof.

4. The use according to the foregoing claim wherein the agonist is used for treatment of progenitor and stem cells prior to transplantation.

5. The use according to one or more of the foregoing claims for the transplantation of hematopoietic progenitor and stem cells, umbilical cord blood and placental stem and progenitor cells, liver stem and progenitor cells (oval cells), mesenchymal stem and progenitor cells, endothelial progenitor cells, skeletal muscle stem and progenitor cells (satellite cells), smooth muscle stem and progenitor cells, intestinal stem and progenitor cells, embryonic stem cells, and genetically modified embryonic stem cells, adult islet/beta stem- and progenitor cell, epidermal progenitor and stem cells, keratinocyte stem cells of cornea, skin and hair follicles, olfactory (bulb) stem and progenitor cells and side population cells from diverse adult tissues.

6. The use according one or more of the foregoing claims to increase the sensitivity of hematopoietic stem cells to SDF-1 induced cellular signals.

7. The use according one or more of the foregoing claims for the treatment of leukemias, lymphoproliferative disorders, aplastic anemia, congenital disorders of the bone marrow, solid tumors, autoimmune disorders, inflammatory diseases, primary immunodeficiencies, primary. systemic amyloidosis, systemic sclerosis, heart diseases, liver diseases, neurodegenerative diseases, multiple sclerosis, M. Parkinson, stroke, spinal cord injury diabetes mellitus, bone diseases, skin diseases, replacement therapy of the skin, retina or cornea, other congenital disorders, vessel diseases like atherosclerosis or cardiovascular disease.

8. A method of improving the successful homing of hematopoietic stem cells by contacting the hematopoietic stem cells in vivo or ex vivo with an agent which is at least one agonist of receptors selected from the group consisting of the CCR3, CCR6 or CCR8 receptor or combinations thereof.

9. A method of improving the successful homing of hematopoietic stem cells in a host patient by applying at least one agent which is an agonist of receptors selected from the group consisting of the CCR3, CCR6 or CCR8 receptor or combinations thereof into the patient who is receiving stem cell transplantation prior to and/or in the course of stem cell transplantation.

10. The method of the foregoing claim wherein the host patient are not conditioned.

11. The method of claim 9 wherein the host patient is conditioned under sublethal, lethal, or supralethal conditions.

12. The method according to any one of the claims 10 or 11 wherein sublethal, lethal, or supralethal conditions include treatment with total body irradiation, optionally followed by treatment with myeloablative Or immunosuppressive agents.

13. The method according to any one of the claims 10 to 12 wherein sublethal, lethal, or supralethal conditions include myeloablative or immunosuppressive treatment without total body irradiation.
